(19) United States
(12) Patent Application Publication
(10) Pub. No.: US 2015/0196271 A1
(43) Pub. Date: Jul. 16, 2015

(54) DETECTING ENDOLEAKS ASSOCIATED WITH ANEURYSM REPAIR

(71) Applicant: VOLCANO CORPORATION, San Diego, CA (US)

(72) Inventor: Anuja Nair, Bedford, MA (US)

(21) Appl. No.: 14/594,599

(22) Filed: Jan. 12, 2015

Related U.S. Application Data
(60) Provisional application No. 61/925,939, filed on Jan. 10, 2014.

(51) Int. Cl. A61B 8/06; A61B 8/08; A61F 2/07; A61B 8/12

(52) U.S. Cl. CPC A61B 8/06; A61B 8/12; A61B 8/5223; A61F 2/07

(57) ABSTRACT

The invention is a system comprising an intravascular imaging probe having expanded imaging capabilities and a processor for processing the image data and causing relevant information, such as flow, to be displayed. The system is configured to cause image data to be processed and reconfigured in a user friendly format, e.g., color-coded, to provide details of flow, including the automated, or semi-automated, detection of endoleaks at or near a placement site of a stent graft within a lumen associated with an EVAR or TEVAR procedure. The system is further configured to provide identification of the type of endoleak present.

### CROSS-REFERENCE TO RELATED APPLICATION

This application claims the benefit of, and priority to, U.S. Provisional Patent Application Ser. No. 61/925,939, filed Jan. 10, 2014, the contents of which are incorporated by reference.

### FIELD OF THE INVENTION

The present invention generally relates to evaluating cardiovascular health, and, more particularly, to methods for detecting endoleaks associated with aneurysm repair using systems having visualization and flow detection capabilities.

### BACKGROUND

An abdominal aortic aneurysm (AAA) is an abnormal swelling of the lower part of the aorta that extends through the abdominal area. The aorta is the primary blood vessel that transports blood from the heart to the rest of the body. The walls of aorta are elastic, which allow the vessel to be filled with blood under high pressure. An aneurysm occurs when the arterial walls become weakened and distended. Many factors can contribute to the weakening of arterial walls, including atherosclerosis, high cholesterol, hypertension, and smoking.

An aneurysm that has become too large may rupture, which is extremely dangerous. Symptoms of a ruptured aneurysm include excruciating pain of the lower back, flank, abdomen and groin. Bleeding associated with the rupture often leads to hypovolemic shock, and if left untreated, will result in a relatively quick death.

Conventional methods of repairing abdominal aortic aneurysms include surgical intervention and minimally invasive procedures like endovascular aneurysm repair (EVAR) and thoracic endovascular aneurysm repair (TEVAR). In the EVAR procedure, a stent graft (also known as an “endograft”) is generally inserted into the aorta through small incisions in the groin. The stent graft reinforces the weakened part of the vessel from the inside and creates a new channel through which the blood flows, eliminating the risk of rupture at the site of aneurysm. A primary concern associated with EVAR is that, despite placement of the stent graft, blood may continue to flow into the aneurysm, in what is commonly known as an endoleak. Endoleaks arising after grafting may be attributed to an incomplete sealing between the stent graft and the aortic wall or defects within the stent graft itself. Endoleaks are the major cause of complications in EVAR and TEVAR procedures, and thus failure in endoluminal treatment of AAA. When an endoleak occurs, it causes continued pressurization of the aneurysm sac and may leave the patient at risk of an AAA rupture and subsequently, immediate death.

Endoleaks are classified based on the origin of blood-leakage. There are generally five types of endoleaks (Type I through Type V). A type I endoleak is a perigraft leakage at proximal or distal stent graft attachment sites (near the renal and iliac arteries), a type II endoleak is a retrograde flow from collateral branches, such as the lumbar and inferior mesenteric arteries. A Type III endoleak is a leakage between overlapping parts of the stent (i.e. connection between overlapping components) or rupture through graft material and a type IV endoleak is leakage through the graft wall, generally due to the quality (porosity) of the graft material. A type V endoleak is generally expansion of the aneurysm sac without an identifiable leak (also called “endotension”).

Type I and Type III leaks are considered to have high risk to the patient and must be identified and fixed during an EVAR/TEVAR procedure. A type I endoleak may be due to mal-apposition, or graft enfolding, of the stent graft at the proximal or distal landing zones (i.e., portions of the stent graft are not touching the luminal wall). A type III endoleak may be due to a damaged stent graft (e.g., hole in graft) or misaligned overlapping segments of the graft, causing a hole in the middle, hence, a leak. If either, or both, of these types of endoleaks are present, the aneurysm will continue to fill with blood and experience high pressure, leading to a high risk of rupture to the patient. As such, the identification and repair of these types of endoleaks is paramount to ensure patient safety and procedural success.

Currently, during EVAR/TEVAR procedures, physicians attempt to detect such leaks with existing imaging techniques, such as external imaging modalities (e.g., angiography, fluoroscopy, computed tomography (CT), and magnetic resonance imaging (MRI))Based on the images, a physician may repair the leaks by correcting the graft-deployment, so as to ensure an appropriate seal. However, the use of non-invasive imaging techniques is restricted to either pre-procedural planning (CT & MRI) or peri-procedural monitoring with known limitations of the incidence-angle (angiography). Additionally, the use of external imaging techniques may be limiting and fail to provide the level of detail that intraluminal imaging techniques are able to offer. As such, the level of detection and monitoring of endoleaks may be sacrificed.

### SUMMARY

The present invention provides systems and methods for detecting endoleaks based on a combination of intraluminal images and functional parameters, such as flow visualization, within the vasculature. In particular, the invention is a system comprising an intravascular diagnostic imaging probe having expanded imaging capabilities and a processor for processing the image data and causing relevant information, such as flow, to be displayed, thereby providing flow visualization. The system is configured to cause image data to be processed and reconfigured in a user friendly format, e.g., color-coded, to provide details of flow and device placement within a biological lumen. The combined flow data and structure images can be particularly useful in evaluating disease states, previously-placed interventional structures, such as, a stent graft, as well as the detection of endoleaks, including the type of endoleak, at or near the site of stent graft placement.

For example, the invention includes an intravascular ultrasound (IVUS) device including an imaging probe having one or more ultrasound transducers, wherein the imaging probe can be introduced into a vessel and maneuvered to the site where the stent graft was placed. Once positioned, the imaging probe can collect the appropriate data, which can then be used to discern the presence of endoleaks. For instance, the ultrasound transducers are configured to acquire more than one form of data related to the vessel and implant placed within. The system is configured to produce an intravascular image that includes not only structure data of the lumen, but further includes intravascular flow data within the lumen, particularly within the site where the endograft was placed.

The system is configured to provide automated detection and identification of different types of endoleaks (Types I through Type IV) based on the flow visualization from the captured image data. In particular, the flow within a lumen can be visualized by a particular color or a pattern of colors corresponding to a particular attribute of the flow (e.g. motion or speed of the flow, direction of the flow, etc.). The system may include the use of phased array imaging, motion detector algorithms, Doppler based signaling, and/or other cross-correlation algorithm used for detecting a change, or a flow pattern, in a series of image and flow data captured by the imaging probe. Additionally, or alternatively, the flow visualization could also be enhanced by use of a contrast material for ultrasound or ultrasound-activated micro bubbles offering flow contrast.

In addition, the imaging probe is configured to capture intravascular image data via ultrasound at a frequency sufficient to provide an adequate field-of-view of the vasculature and surrounding tissues. For example, in one embodiment, the imaging probe is configured to capture the intravascular image data via ultrasound at a frequency between at least 9 MHz and 11 MHz, so as to provide near 360 degree tomographic views of the vascular walls from within the lumen. Accordingly, the probe consistent with the present disclosure is operable to capture image data providing adequate field-of-view of the large aorta, for example.

Systems and methods of the present invention will improve interventional evaluation by providing a physician with critical information about flow and structure while also reducing the time for procedures. In particular, the addition of flow visualization capability to IVUS can significantly improve the detection of endoleaks. More specifically, IVUS can provide accurate measurements in sizing and assessing stent graft landing zones, which is generally more accurate than the traditional external imaging modalities (e.g., angiography, CT, MRI, etc.). Furthermore, with flow visualization and identification of endoleaks, physicians would not have to rely heavily on the use of angiography, thereby reducing harmful radiation and contrast exposure to the patient. In further embodiments, the system is useful in the various clinical scenarios, such as use for identifying vascular dissections and true lumen (to ensure stents and grafts are deployed in the true lumen), detection of thrombus in the lumen at the site of an AAA, as well as venous thrombus identification within.

Systems and methods of the invention are useful in verifying the effectiveness of EVAR/TEVAR procedures. Exclusion of the aneurysm sac is the main goal of the stent graft treatment, and clinical success is defined by the “total exclusion” of the aneurysm. By confirming the absence of endoleaks using the provided methods, the aneurysm can be deemed to have been totally excluded. In addition, the early detection of endoleaks, as well as the type of endoleak, at the time of surgery (perioperatively) can avoid complications at a later time and decrease patient mortality.

### BRIEF DESCRIPTION OF THE DRAWINGS

FIG. 1 is a schematic drawing showing an illustrative embodiment of an IVUS medical system in a catheterization laboratory.

FIG. 2 is a schematic drawing illustrating an IVUS catheter consistent with one embodiment of the present invention.

FIG. 3A depicts a distal end of another embodiment of an intravascular imaging device including a guidewire having a pressure sensor integrated into the distal tip.

FIG. 3B depicts the simultaneous or sequential delivery and reception of acoustic waves (curved lines) from the distal end of the guidewire of FIG. 3A.

FIG. 4 depicts an embodiment of a system for ultrasound imaging with the IVUS catheter of FIG. 2 and display of flow and structural information acquired by the IVUS transducer elements.

FIG. 5 is a block diagram of an exemplary system for identifying flow and/or structure in an image acquired with an imaging guidewire and displaying relevant flow and/or structure information.

FIG. 6 is a rendition of the block diagram of FIG. 5 depicting how data can be stored and transferred over a hospital (internal and external) network and then accessed by a user via a separate terminal also connected to the same network.

FIG. 7 is a block diagram of an exemplary system for assessing, analyzing, and transforming data acquired by an intravascular imaging probe consistent with the present invention.

FIG. 8 is a cross-sectional view of a portion of an aorta illustrating placement of stent graft at an abdominal aortic aneurysm (AAA) site during an EVAR procedure.

FIG. 9 is a side view of the aorta of FIG. 8 illustrating positioning of the IVUS catheter of FIG. 2 for capturing image and flow data related to at least the stent graft and aortic wall for providing at least flow visualization.

FIGS. 10-13 show various gray-scale IVUS images of vessels provided by a system consistent with the present disclosure.

FIG. 14A is a gray-scale IVUS image of a vessel, FIG. 14B is an image of flow within the vessel, and FIG. 14C is a composite image of the flow data overlaid on the gray-scale image provided by a system consistent with the present invention.

### DETAILED DESCRIPTION

The present invention provides systems and methods for detecting endoleaks based on a combination of intraluminal images and functional parameters, such as flow, within the vasculature. In particular, the invention is a system comprising an intravascular diagnostic imaging probe having expanded imaging capabilities and a processor for processing the image data and causing relevant information, such as flow, to be displayed, thereby providing flow visualization. In one embodiment, the invention includes an intravascular ultrasound (IVUS) device including an imaging probe having one or more ultrasound transducers, wherein the imaging probe can be introduced into a vessel and maneuvered to site where the stent graft was placed. Once positioned, the imaging probe can collect the appropriate data, which can then be used to discern the presence of endoleaks. For instance, the ultrasound transducers are configured to acquire more than one form of data related to the vessel and stent placed within. The system is configured to produce an intravascular image that includes not only structure data of the lumen, but further includes intravascular flow data within the lumen, particularly within the site where the stent graft was placed.

In a further embodiment, the system is useful for evaluating intravascular structures to determine the placement of the structure and the efficacy, e.g., stent graft for AAA. The system is further configured to provide automated or semi-automated detection, as well as classification, of endoleaks at or near the placement site of the stent graft based on flow visualization. The disclosed invention will improve interventional evaluation by providing a physician with critical information about flow and structure while also reducing the time for procedures.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer.

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector. While aspects of the invention are described in relation to solid-state IVUS devices, one of skill in the art will recognize that the invention also applies to rotational IVUS devices.

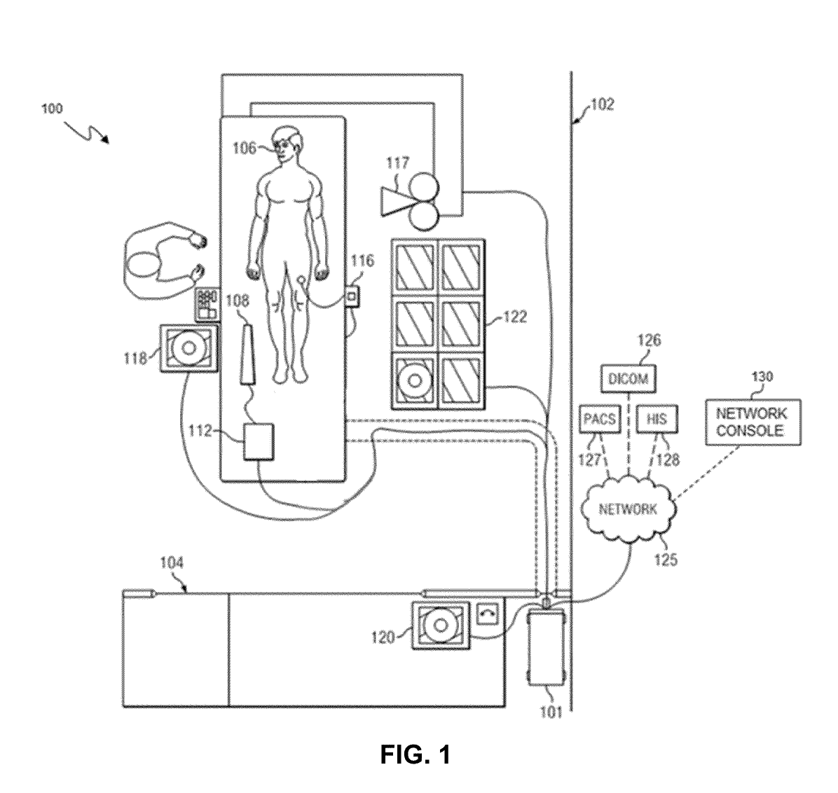

FIG. 1 is a schematic drawing depicting a medical system including an IVUS imaging system in various applications according to some embodiments of the present disclosure. In general, the medical system 100 may be a single modality medical system, such as an IVUS system, and may also be a multi-modality medical system. In that regard, a multi-modality medical system provides for coherent integration and consolidation of multiple forms of acquisition and processing elements designed to be sensitive to a variety of methods used to acquire and interpret human biological physiology and morphological information and coordinate treatment of various conditions.

With reference to FIG. 1, the imaging system 101 is an integrated device for the acquisition, control, interpretation, and display of one or more modalities of medical sensing data. Accordingly, in some embodiments, the imaging system 101 is a single modality imaging system, such as an IVUS imaging system, whereas, in some embodiments, the imaging system 101 is a multi-modality imaging system. In one embodiment, the imaging system 101 includes a computer system with the hardware and software to acquire, process, and display medical imaging data, but, in other embodiments, the imaging system 101 includes any other type of computing system operable to process medical data. In the embodiments in which the imaging system 101 includes a computer workstation, the system includes a processor such as a microcontroller or a dedicated central processing unit (CPU), a non-transitory computer-readable storage medium such as a hard drive, random access memory (RAM), and/or compact disk read only memory (CD-ROM), a video controller such as a graphics processing unit (GPU), and/or a network communication device such as an Ethernet controller and/or wireless communication controller. In that regard, in some particular instances, the imaging system 101 is programmed to execute steps associated with the data acquisition and analysis described herein.

Accordingly, it is understood that any steps related to data acquisition, data processing, instrument control, and/or other processing or control aspects of the present disclosure may be implemented by the imaging system 101 using corresponding instructions stored on or in a non-transitory computer readable medium accessible by the processing system. In some instances, the imaging system 101 is portable (e.g., handheld, on a rolling cart, etc.). Further, it is understood that in some instances imaging system 101 includes a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

In the illustrated embodiment, the medical system 100 is deployed in a catheter lab 102 having a control room 104, with the imaging system 101 being located in the control room. In other embodiments, the imaging system 101 may be located elsewhere, such as in the catheter lab 102, in a centralized area in a medical facility, or at an off-site location accessible over a network. For example, the imaging system 101 may be a cloud-based resource. The catheter lab 102 includes a sterile field generally encompassing a procedure area, whereas the associated control room 104 may or may not be sterile depending on the requirements of a procedure and/or health care facility. The catheter lab and control room may be used to perform on a patient any number of medical sensing procedures such as intravascular ultrasound (IVUS), angiography, virtual histology (VH), forward looking IVUS (FL-IVUS), intravascular photoacoustic (IVPA) imaging, a fractional flow reserve (FFR) determination, a coronary flow reserve (CFR) determination, optical coherence tomography (OCT), computed tomography (CT), intracardiac echocardiography (ICE), forward-looking ICE (FLICE), intravascular palpography, transesophageal ultrasound (TEE), thermography, magnetic resonance imaging (MRI), micro-magnetic resonance imaging (mMRI or μMRI), or any other medical sensing modalities known in the art. Further, the catheter lab and control room may be used to perform one or more treatment or therapy procedures on a patient such as radiofrequency ablation (RFA), cryotherapy, atherectomy or any other medical treatment procedure known in the art. For example, in catheter lab 102 a patient 106 may be undergoing a multi-modality procedure either as a single procedure or multiple procedures. In any case, the catheter lab 102 includes a plurality of medical instruments including medical sensing devices that collects medical sensing data in various different medical sensing modalities from the patient 106.

In the illustrated embodiment of FIG. 1, instrument 108 is a medical sensing device that may be utilized by a clinician to acquire medical sensing data about the patient 106. For instance, the instrument may collect one of pressure, flow (velocity), images (including images obtained using ultrasound (e.g., IVUS), OCT, thermal, and/or other imaging techniques), temperature, and/or combinations thereof. In some embodiments, device 108 collects medical sensing data in different versions of similar modalities. For example, in one such embodiment, device 108 collects pressure data and image data. In another such embodiment, device 108 collects 10 MHz IVUS data, 20 MHz IVUS data, or 40 MHz IVUS data. Accordingly, the device 108 may be any form of device, instrument, or probe sized and shaped to be positioned within a vessel, attached to an exterior of the patient, or scanned across a patient at a distance.

In the illustrated embodiment of FIG. 1, instrument 108 is an IVUS catheter 108 that may include one or more sensors such as a phased-array transducer to collect IVUS sensing data. In some embodiments, the IVUS catheter 108 may be capable of multi-modality sensing such as image and flow sensing. In some instances, an IVUS patient interface module (PIM) 112 is coupled to the IVUS catheter 108, which is coupled to the imaging system 101. In particular, the IVUS PIM 112 is operable to receive medical sensing data collected from the patient 106 by the IVUS catheter 108 and is operable to transmit the received data to the imaging system 101 in the control room 104. In one embodiment, the PIM 112 includes analog to digital (A/D) converters and transmits digital data to the imaging system 101. However, in other embodiments, the PIM transmits analog data to the processing system. In one embodiment, the IVUS PIM 112 transmits the medical sensing data over a Peripheral Component Interconnect Express (PCIe) data bus connection, but, in other embodiments, it may transmit data over a USB connection, a Thunderbolt connection, a FireWire connection, an Ethernet connection, or some other high-speed data bus connection. In other instances, the PIM may be connected to the imaging system 101 via wireless connections using IEEE 802.11 Wi-Fi standards, Ultra Wide-Band (UWB) standards, wireless FireWire, wireless USB, or another high-speed wireless networking standard.

Additionally, in the medical system 100, an electrocardiogram (ECG) device 116 is operable to transmit electrocardiogram signals or other hemodynamic data from patient 106 to the imaging system 101. Further, an angiogram system 117 is operable to collect x-ray, computed tomography (CT), or magnetic resonance images (MRI) of the patient 106 and transmit them to the imaging system 101. In one embodiment, the angiogram system 117 is communicatively coupled to the processing system of the imaging system 101 through an adapter device. Such an adaptor device may transform data from a proprietary third-party format into a format usable by the imaging system 101. In some embodiments, the imaging system 101 is operable to co-register image data from angiogram system 117 (e.g., x-ray data, MRI data, CT data, etc.) with sensing data from the IVUS catheter 108. As one aspect of this, the co-registration may be performed to generate three- and four-dimensional images with the sensing data.

A bedside controller 118 is also communicatively coupled to the imaging system 101 and provides user control of the particular medical modality (or modalities) being used to diagnose the patient 106. In the current embodiment, the bedside controller 118 is a touch screen controller that provides user controls and diagnostic images on a single surface. In alternative embodiments, however, the bedside controller 118 may include both a non-interactive display and separate controls such as physical buttons and/or a joystick. In the integrated medical system 100, the bedside controller 118 is operable to present workflow control options and patient image data in graphical user interfaces (GUIs). In some embodiments, the bedside controller 118 includes a user interface (UI) framework service through which workflows associated with multiple modalities may execute. Thus, the bedside controller 118 may be capable of displaying workflows and diagnostic images for multiple modalities allowing a clinician to control the acquisition of multi-modality medical sensing data with a single interface device.

A main controller 120 in the control room 104 is also communicatively coupled to the imaging system 101 and, as shown in FIG. 1, is adjacent to catheter lab 102. In the current embodiment, the main controller 120 is similar to the bedside controller 118 in that it includes a touch screen and is operable to display a multitude of GUI-based workflows corresponding to different medical sensing modalities via a UI framework service executing thereon. In some embodiments, the main controller 120 is used to simultaneously carry out a different aspect of a procedure's workflow than the bedside controller 118. In alternative embodiments, the main controller 120 includes a non-interactive display and standalone controls such as a mouse and keyboard.

The medical system 100 further includes a boom display 122 communicatively coupled to the imaging system 101. The boom display 122 may include an array of monitors, each capable of displaying different information associated with a medical sensing procedure. For example, during an IVUS procedure, one monitor in the boom display 122 may display a tomographic view and one monitor may display a sagittal view.

Further, the multi-modality imaging system 101 is communicatively coupled to a data network 125. In the illustrated embodiment, the data network 125 is a TCP/IP-based local area network (LAN); however, in other embodiments, it may utilize a different protocol such as Synchronous Optical Networking (SONET), or may be a wide area network (WAN). The imaging system 101 may connect to various resources via the network 125. For example, the imaging system 101 may communicate with a Digital Imaging and Communications in Medicine (DICOM) system 126, a Picture Archiving and Communication System (PACS) 127, and a Hospital Information System (HIS) 128 through the network 125. Additionally, in some embodiments, a network console 130 may communicate with the imaging system 101 via the network 125 to allow a doctor or other health professional to access the aspects of the medical system 100 remotely. For instance, a user of the network console 130 may access patient medical data such as diagnostic images collected by imaging system 101, or, in some embodiments, may monitor or control one or more on-going procedures in the catheter lab 102 in real-time. The network console 130 may be any sort of computing device with a network connection such as a PC, laptop, smartphone, tablet computer, or other such device located inside or outside of a health care facility.

Additionally, in the illustrated embodiment, medical sensing tools in system 100 discussed above are shown as communicatively coupled to the imaging system 101 via a wired connection such as a standard copper link or a fiber optic link, but, in alternative embodiments, the tools may be connected to the imaging system 101 via wireless connections using IEEE 802.11 Wi-Fi standards, Ultra Wide-Band (UWB) standards, wireless FireWire, wireless USB, or another high-speed wireless networking standard.

One of ordinary skill in the art would recognize that the medical system 100 described above is simply an example embodiment of a system that is operable to collect diagnostic data associated with a plurality of medical modalities. In alternative embodiments, different and/or additional tools may be communicatively coupled to the imaging system 101 so as to contribute additional and/or different functionality to the medical system 100.

As mentioned previously, in some embodiments, the imaging assembly is an IVUS imaging assembly. The imaging assembly can be a phased array IVUS imaging assembly, a pull-back type IVUS imaging assembly, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et al., U.S. Pat. No. 4,917,097, Eberle et al., U.S. Pat. No. 5,135,486, and other references well known in the art relating to intraluminal ultrasound devices and modalities. All of these references are incorporated by reference herein.

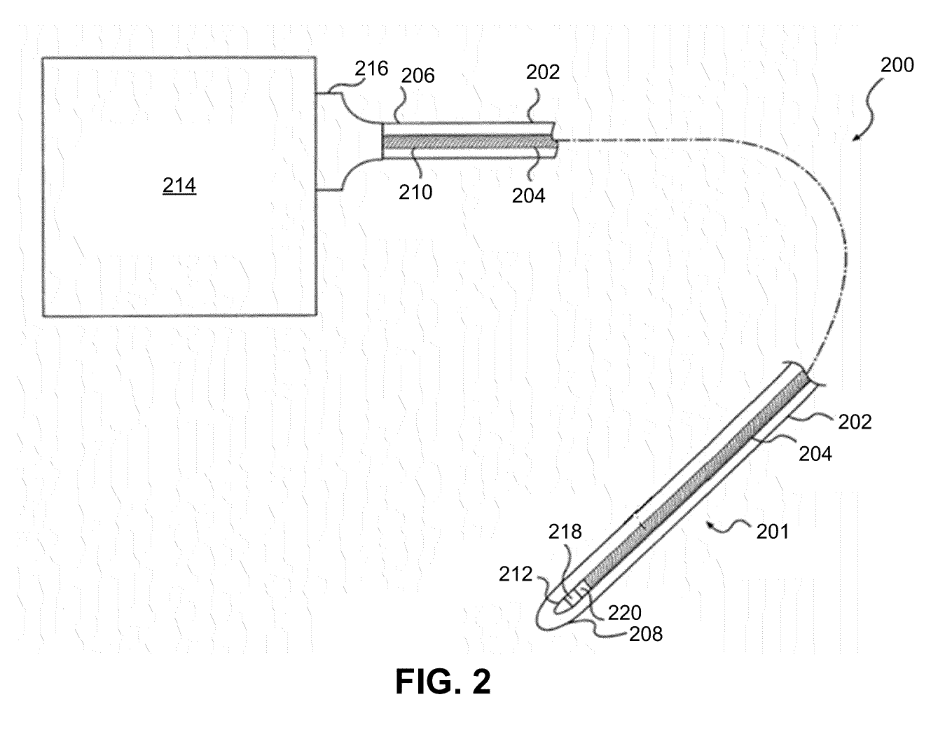

FIG. 2 shows one embodiment of an intravascular imaging probe 200 for insertion into a patient for diagnostic imaging. The probe 200 of FIG. 2 is a solid-state intravascular ultrasound probe 200. However, it should be noted that other embodiments consistent with the present disclosure may include rotational IVUS devices. The probe 200 includes a catheter 201 having a catheter body 202 and a hollow transducer shaft 204. The catheter body 202 is flexible and has both a proximal end portion 206 and a distal end portion 208. The catheter body 202 may be a single lumen polymer extrusion, for example, made of polyethylene (PE), although other polymers may be used. Further, the catheter body 202 may be formed of multiple grades of PE, for example, HDPE and LDPE, such that the proximal portion exhibits a higher degree of stiffness relative to the mid and distal portions of the catheter body. This configuration provides an operator with catheter handling properties required to efficiently perform the desired procedures.

The catheter body 202 is a sheath surrounding the transducer shaft 204. For explanatory purposes, the catheter body 202 in FIG. 2 is illustrated as visually transparent such that the transducer shaft 204 disposed therein can be seen, although it will be appreciated that the catheter body 202 may or may not be visually transparent. The transducer shaft 204 may be flushed with a sterile fluid, such as saline, within the catheter body 202. A fluid injection port (not shown) may be supplied at a junction of the catheter body 202 to the interface module so that the space inside the catheter body 202 can be flushed initially and periodically. The fluid serves to eliminate the presence of air pockets around the transducer shaft 204 that adversely affect image quality. The transducer shaft 204 has a proximal end portion 210 disposed within the proximal end portion 206 of the catheter body 202 and a distal end portion 212 disposed within the distal end portion 208 of the catheter body 202.

The distal end portion 208 of the catheter body 202 and the distal end portion 212 of the transducer shaft 204 are inserted into a patient. The usable length of the probe 200 (the portion that can be inserted into a patient) can be any suitable length and can be varied depending upon the application. The distal end portion 212 of the transducer shaft 204 includes a transducer subassembly 218.

The transducer subassembly 218 is used to obtain ultrasound information from within a vessel. It will be appreciated that any suitable frequency and any suitable quantity of frequencies may be used. Exemplary frequencies range from about 5 MHz to about 80 MHz. In certain embodiments, the IVUS transducers operate at 10 MHz, or 20 MHz. In some embodiments, a frequency less than 20 MHz, such as between 9 and 11 MHz, and 10 MHz is used. Generally, lower frequency information (e.g., less than 40 MHz) facilitates a tissue versus blood classification scheme due to the strong frequency dependence of the backscatter coefficient of the blood. Higher frequency information (e.g., greater than 40 MHz) generally provides better resolution at the expense of poor differentiation between blood and tissue, which can make it difficult to identify the vessel-lumen border. Flow detection algorithms, including motion-detection algorithms (such as CHROMAFLO (IVUS fluid flow display software; Volcano Corporation), Q-Flow, B-Flow, Delta-Phase, Doppler, Power Doppler, etc.), temporal algorithms, harmonic signal processing, can be used to differentiate blood speckle from other structural tissue, and therefore enhance images where ultrasound energy back scattered from blood causes image artifacts.

The catheter body 202 may include a flexible atraumatic distal tip. For example, an integrated distal tip can increase the safety of the catheter by eliminating the joint between the distal tip and the catheter body. The integral tip can provide a smoother inner diameter for ease of tissue movement into a collection chamber in the tip. During manufacturing, the transition from the housing to the flexible distal tip can be finished with a polymer laminate over the material housing. No weld, crimp, or screw joint is usually required. The atraumatic distal tip permits advancing the catheter distally through the blood vessel or other body lumen while reducing any damage caused to the body lumen by the catheter. Typically, the distal tip will have a guidewire channel to permit the catheter to be guided to the target lesion over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip.

The interface module 214 communicates with the transducer subassembly 218 by sending and receiving electrical signals to and from the transducer subassembly 218 via at least one electrical signal transmission member (e.g., wires or coaxial cable) within the transducer shaft 204. The interface module 214 can receive, analyze, and/or display information received through the transducer shaft 204. It will be appreciated that any suitable functionality, controls, information processing and analysis, and display can be incorporated into the interface module 214. Further description of the interface module is provided, for example in Corl (U.S. patent application number 2010/0234736).

The transducer shaft 204 includes a transducer subassembly 218 and a transducer housing 220. The transducer subassembly 218 is coupled to the transducer housing 220. The transducer housing 220 is located at the distal end portion 212 of the transducer shaft 204. The transducer subassembly 218 can be of any suitable type, including but not limited to one or more advanced transducer technologies such as PMUT or CMUT.

The transducer subassembly 218 can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLO (IVUS fluid flow display software; Volcano Corporation).

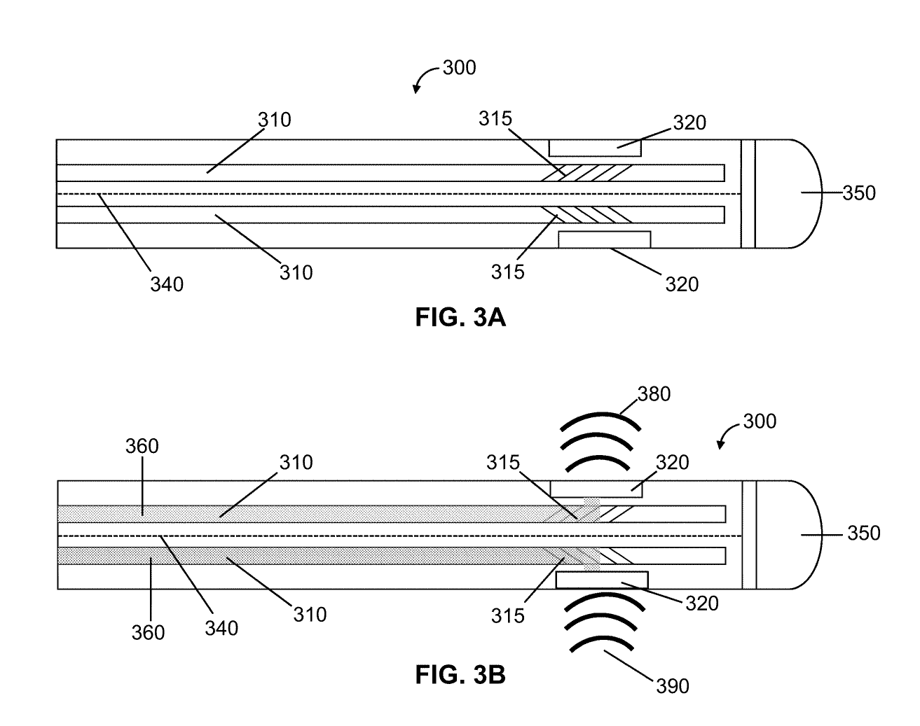

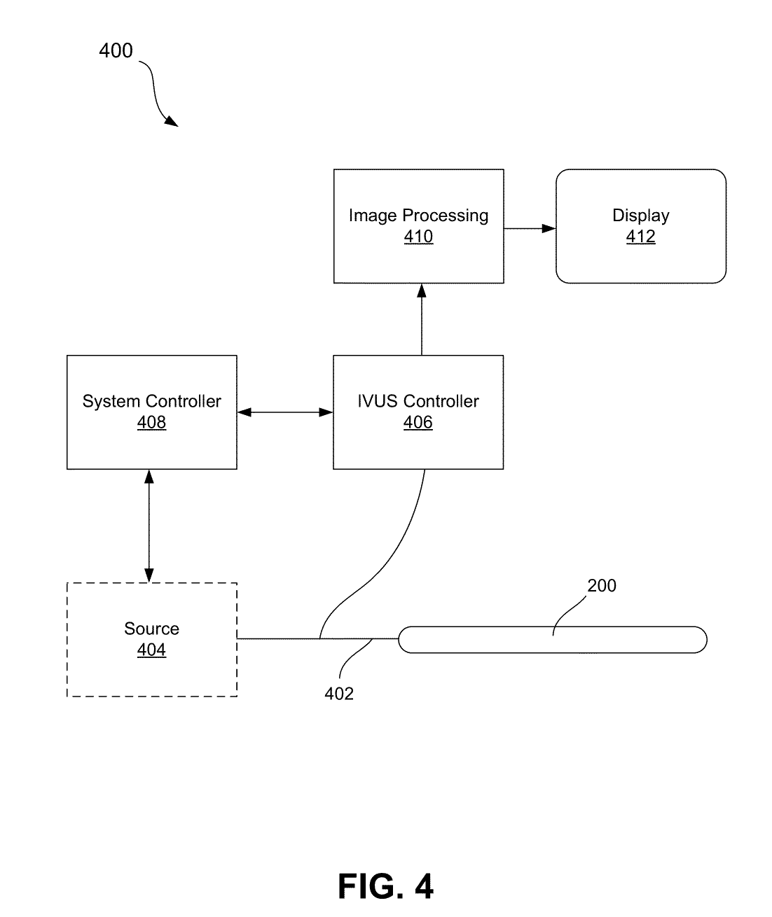

FIG. 3A depicts a distal end of another embodiment of an intravascular imaging device including a guidewire for acquiring data related to a vessel and a stent placed therein, wherein such data can be used to discern the presence and type of endoleaks. Guidewires typically have diameters of 0.010″ to 0.035″, with 0.014″ being the most common. Guidewires (and other intravascular objects) are also sized in units of French, each French being ⅓ of a mm or 0.013″. Guidewire lengths vary up to 600 cm, depending on the anatomy and work flow. The ends of the guidewire are denoted as distal (far from the user, i.e., inside the body) and proximal (near the user, i.e., outside the body). Often a guidewire has a flexible distal tip portion about 3 cm long and a slightly less flexible portion about 30 to 50 cm long leading up to the tip with the remainder of the guidewire being stiffer to assist in maneuvering the guidewire through tortuous vasculature, etc. The tip of a guidewire typically has a stop or a hook to prevent a guided device, e.g., a catheter from passing beyond the distal tip. In some embodiments, the tip can be deformed by a user to produce a desired shape.

Advanced guidewire designs include sensors that measure flow and pressure, among other things. For example, the FLOWIRE® Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PRIMEWIRE® pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety.

The proximal end of a guidewire varies depending upon the complexity of the device. Simple guidewires, used for placement of devices such as catheters, are untethered, i.e., the proximal end does not need to be connected to other equipment. Sensing guidewires, on the other hand, require a signal connection when the sensor is used. The signal connection is typically detachable to facilitate loading/unloading catheters, however it is also possible to load a rapid exchange catheter on a guidewire prior to guidewire insertion. Placement guidewires without tethers are less expensive, and most useful when a procedure requires multiple catheter exchanges, because each catheter can be quickly removed from the guidewire and the next catheter placed on the guidewire.

While not shown in detail in the figures, a sensing guidewire has a tethered proximal end, typically with a detachable connection. As discussed below, guidewires of the invention use optical fibers to supply light to the distal end of the guidewire and to detect returning light. Accordingly, guidewires of the invention have a tether comprising optical fibers and one or more detachable optical couplings. In some embodiments, all of the optical fibers of the guidewire are coupled into a single optical coupling. The tethers may additionally comprise electrical connections, as needed, to produce acoustic energy or to receive acoustic echoes.

Additionally, while not shown in detail in the figures, a guidewire of the invention has a mid-body connecting the proximal and distal ends. The mid-body is typically a length between 50 and 500 cm, typically greater than or equal to 100 cm, typically less than or equal to 600 cm, typically about 200 to 500 cm. The mid-body typically has a core, which is typically a biocompatible and resilient metal wire. The core may comprise multiple strands of metal fiber or the core may be a unitary piece of metal wire. The core is typically constructed from nitinol or stainless steel. As discussed in greater detail below, the mid-body will also comprise a number of optical fibers to deliver light to the distal end of the guidewire and to return reflected light. The optical fibers may be bound to the core with adhesive or fasteners. The optical fibers may be touching the core or the optical fibers may be displaced axially from the core with spacer, typically a resilient polymer. The core and the optical fibers (and optionally spacer) are coated with a coating to help the guidewire pass through an introducer, to pass through the vasculature, and to help delivered devices (e.g., catheter) easily pass over the guidewire. In addition to being both biocompatible and resilient (will not dislodge or flake), the guidewire coating is typically lubricious to reduce the friction between the guidewire and a catheter.

The sensors incorporated into a guidewire of the invention can be of a variety of structures small enough to be incorporated into a guidewire and suitable for pressure sensing in an anatomical environment, e.g., an artery or vein. A guidewire mounted pressure sensor may be, for example, a MEMS sensor manufactured using deep reactive ion etching (DRIE) to form the solid-state sensor rather than previously used mechanical saws. DRIE is a highly anisotropic etch process for creating deep, steep-sided holes and trenches in solid-state device wafers, with aspect ratios of 20:1 or more. DRIE was originally developed for MEMS structures such as cantilever switches and microgears. However, DRIE is also used for producing other devices such as to excavate trenches for high-density capacitors for DRAM. DRIE is capable of fabricating 90° (truly vertical) walls. Using DRIE leads to a number of new pressure sensor designs for intravascular applications wherein the sensor is mounted at a distal end of a pressure measuring coronary guidewire.

A distal end of an embodiment of a guidewire 300 suitable for use in a system of the invention is depicted in FIG. 3A. The guidewire 300 comprises optical fibers 310. Optical fibers 310 may be constructed from glass or plastic. Optical fibers 310 include blazed Bragg gratings 315 (discussed below). In the embodiment shown in FIG. 3A, the blazed Bragg grating 315 of the optical fiber 310 is in proximity to an ultrasound transducer 320. The ultrasound transducer 320 may also comprise a photoreflective element that is deflected with the receipt of incident acoustic waves. In other embodiments, the ultrasound transducer and photoreflective elements are separate structures, however it is to be understood that ultrasound transducer 320 refers to a stand-alone ultrasound transducer, a combined ultrasound transducer and photoreflective element, or a stand-alone photoreflective element. The guidewire 300 terminates in a tip 350. The core of the guidewire is not shown in FIG. 3A to assist clarity, however, acorn is typically present in the guidewire 300, as discussed above.

The guidewires of the invention employ fiber Bragg gratings to couple light into or out of the optical fibers 310. A fiber Bragg grating is a periodic modulation of the index of refraction in a fiber. When the periodicity, d, of the modulation satisfies the Bragg condition (d=nλ/2) for a wavelength λ, that wavelength will be reflected. That is, the fiber Bragg grating acts as a wavelength-selective mirror. The degree of index change and the length of the grating influences the ratio of light reflected to that transmitted through the grating. A review of fiber Bragg gratings can be found at A. Othonos, Rev. Sci. Inst., 68 (12), 4309 (1997), incorporated by reference herein in its entirety. The optical fibers 310 comprise a normal Bragg grating (back reflective—not shown in FIG. 3A) in addition to blazed Bragg gratings (angle reflective) 315. Blazed Bragg gratings are discussed in greater detail in Othonos, referenced above.

As shown in FIG. 3B, the blazed Bragg gratings couple light, 360, from the optical fibers 310, out of the fibers and into an ultrasound transducer 320. The light 360 originates in a light source, discussed in detail below. As shown in FIG. 3B, the light 360 coupled out of the first optical fiber 310 by the blazed Bragg grating 315 will impinge on the ultrasound transducer 320 producing outbound ultrasonic waves 380. The outbound ultrasonic waves 380 are then absorbed, reflected, and scattered by the tissues surrounding the ultrasonic transducer 320. The inbound ultrasonic waves 390, i.e., reflected, etc. are received by the ultrasonic transducer 320, resulting in a deflection of photoreflective materials (not shown). The change in a pathlength between the photoreflective material and the blazed Bragg grating results in a signal that can be used to image the tissue surrounding the device (discussed in detail below). In some embodiments, a similar structure of blazed Bragg gratings 315 and ultrasonic transducers 320 can be used to make Doppler measurements, e.g., of a flowing fluid, e.g., blood.

The ultrasound transducer 320 comprises an optically-absorptive photoacoustic material, which produces ultrasound waves 380 when it absorbs light 360. The optically absorptive photoacoustic material is positioned, with respect to the blazed Bragg grating 315, to receive the optical energy leaving the blazed grating. The optically absorptive photoacoustic material is selected to absorb light 360, and produce and transmit ultrasound or other acoustic waves for acoustic imaging of a region of interest about the distal tip of the guidewire 300. The acoustic waves generated by the photoacoustic material interact with tissues (e.g., vasculature) in the vicinity of the distal end of the guidewire 300, and are reflected back (echoes). The reflected acoustic waves are collected and analyzed to obtain information about the distance from the tissues to the guidewire, or the type of tissue, or other information, such as blood flow or pressure.

As discussed above, the ultrasound transducer 320 may comprise a photoreflective element to receive reflected acoustic waves. The photoreflective member is flexibly resilient, and is displaced by acoustic waves reflected by the tissues. A transparent (or translucent) flexible material is disposed between the optical fiber 310 and the photoreflective material of the ultrasound transducer 320, thereby allowing a deflection in the photoreflective material to change the path length of the light between the optical fiber 310 and the photoreflective material. In alternative embodiments, a void can be left between the optical fiber 310 and the photoreflective material.

In the absence of incident acoustic energy, the photoreflective material will be in a neutral position, providing a baseline path length between the optical fiber 310 and the photoreflective material. Incident light, transmitted via the optical fiber 310, will be reflected from the photoreflective material, and return to a detector at the proximal end of guidewire (not shown) with a characteristic round trip time. The light transmitted via the optical fiber 310 may be the same light as used to produce acoustic energy (discussed above), the same light used to photoactivate therapeutics (discussed above), or a different light (wavelength, pulse frequency, etc.) may be used. When the photoreflective material is deflected, i.e., with the absorbance of incident acoustic waves, the path length between the third optical fiber 310 and the photoreflective material will change, resulting in a measurable change in the properties of the reflected light, as measured by a detector at the proximal end of guidewire (not shown). The change may be a shift in the time of the return trip, or the shift may be an interferometric measurement. The change in the properties of the reflected light can then be analyzed to determine properties of the tissues from which the acoustic waves were reflected.

In some embodiments, the incident light 360 is pulsed at a frequency at which the acoustic waves will be produced. Light sources that produce pulses at ultrasonic frequencies, e.g., 1 MHz and greater, are commercially-available, typically solid state lasers. Nonetheless, photoacoustic materials have natural acoustic resonances, and the photoacoustic material will naturally produce a spectrum of acoustic frequencies when the material absorbs the incident light 360 and the photoacoustic material relaxes by producing acoustic waves. If it is desired to rely on the natural frequencies of the photoacoustic material, the incident light 160 may be continuous.

In an embodiment, the photoacoustic material has a thickness in the direction of propagation that increases the efficiency of emission of acoustic energy. In some embodiments, the thickness of the photoacoustic material is selected to be about one fourth of the acoustic wavelength of the material at the desired acoustic frequency (“quarter wave matching”). Providing photoacoustic material with quarter wave matching improves the generation of acoustic energy by the photoacoustic material, resulting in improved ultrasound images. Using the quarter wave matching and sensor shaping techniques, the productivity of the fiber blazed Bragg sensor and photoacoustic materials approaches the productivity of piezoelectric transducers known in the field of ultrasound imaging.

In one embodiment, before the photoacoustic transducer is fabricated, the guidewire 300 is assembled, such as by binding the optical fibers 310 to the core (not shown) and tip 150, and optionally coating the guidewire 300. The photoacoustic transducer 320 is then integrated into the guidewire 300 by etching or grinding a groove in the assembled guidewire 300 above the intended location of the blazed Bragg grating 315 in the first optical fiber 310. As discussed above, the depth of the groove in the assembled guidewire 300 can play a role in the efficiency of the acoustic wave production (e.g., quarter wave matching).

After the photoacoustic transducer 320 location has been defined, the blazed Bragg grating 315 can be added to the first optical fiber 310. In one example, the grating 315 is created using an optical process in which the portion of the first optical fiber 310 is exposed to a carefully controlled pattern of UV radiation that defines the blazed Bragg grating 315. After the blazed Bragg grating is complete, a photoacoustic material is deposited or otherwise added over the blazed Bragg grating 315 to complete the transducer 320. An exemplary photoacoustic material is pigmented polydimethylsiloxane (PDMS), such as a mixture of PDMS, carbon black. and toluene. The photoacoustic materials may naturally absorb the light 360, or the photoacoustic material may be supplemented with dyes, e.g., organic dyes, or nanomaterials (e.g., quantum dots) that absorb light 360 strongly. The photoacoustic material can also be “tuned” to selectively absorb specific wavelengths by selecting suitable components.

In another embodiment, not shown in the figures, the optical fibers 310 may be modified to include first and second normal Bragg gratings. These first and second normal Bragg gratings are partially and mostly reflective, respectively, and create a resonant cavity in the optical fiber 310. In the absence of incident acoustic energy, light in the resonant cavity has a characteristic return signature, e.g., an optical decay signal. With the incidence of reflected acoustic energy, the path length and/or path direction of the resonant cavity will be modified, leading to a change in the return signature. By monitoring changes in the return signature, it is possible to determine the timing of reflected acoustic signals, and hence, properties of the tissues from which the acoustic waves were reflected. The detection is similar to known methods of detecting strain or temperature changes with optical fibers.

In one example of operation of this alternate embodiment, light reflected from the blazed Bragg grating 315 excites the photoacoustic material 320 in such a way that the optical energy is efficiently converted to substantially the same acoustic frequency for which the resonant cavity sensor is designed. The blazed Bragg grating 315 and the photoacoustic material 320, in conjunction with the resonant sensor, provide both an acoustic transducer and a receiver, which are harmonized to create an efficient unified optical-to-acoustic-to-optical transmit/receive device. In some embodiments, more than one type of light (e.g., wavelength) can be coupled into the same fiber, allowing one to be used to produce the acoustic wave and another to monitor reflected acoustic waves. In a further example, the optical transmit/receive frequencies are sufficiently different that the reception is not adversely affected by the transmission, and vice-versa.

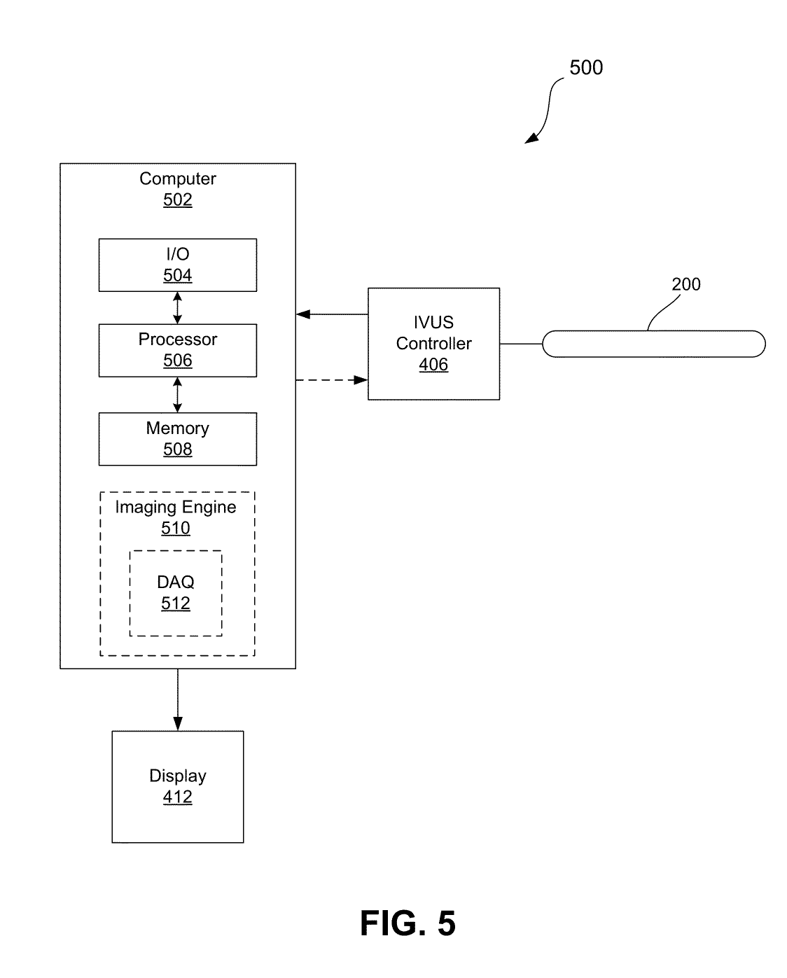

The intravascular imaging devices described herein may be used as part of a system for imaging and identifying flow and structures within a vessel. An exemplary system 400 is shown in FIG. 4. The system 400 may generally refer to the IVUS imaging system 101 described with respect to FIG. 1. Accordingly, the system 400 is shown to include the IVUS imaging probe 200 of FIG. 2. It should be noted that in other embodiments consistent with the present disclosure, the system 400 may include other intravascular image devices described herein, such as the guidewire 300 of FIGS. 3A and 3B. As such, the system may optionally include a source of light 404 to be coupled into an optical fiber 402 of a guidewire 300 and capable of producing light with the desired temporal and frequency characteristics.

As shown, the system 400 includes an IVUS controller 406 for controlling functionality of the IVUS probe 200, including operation of the transducer elements in the different modes (e.g., gray-scale and flow imaging modes) as described herein. The system 400 further includes a system controller 408 that may control timing, duration, and amount of imaging. The system controller 408 is further interfaced with image processing 410. The image processing 410 may be configured to construct IVUS images based on the data sets acquired by the IVUS transducer elements related to the structure and flow of the vessel. The image processing 410 may additionally include spectral analysis, i.e., examining the energy of the returned acoustic signal at various frequencies. Spectral analysis is useful for determining the nature of the tissue and the presence of foreign objects. A plaque deposit, for example, will typically have a different spectral signature than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. Other signal processing techniques known in the art of tissue characterization may also be applied.

Other image processing may facilitate use of the images or identification of features of interest. For example, the border of a lumen may be highlighted or plaque deposits may be displayed in a visually different manner (e.g., by assigning plaque deposits a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. Other measurements, such as flow rates or pressure may be displayed using color mapping or by displaying numerical values.

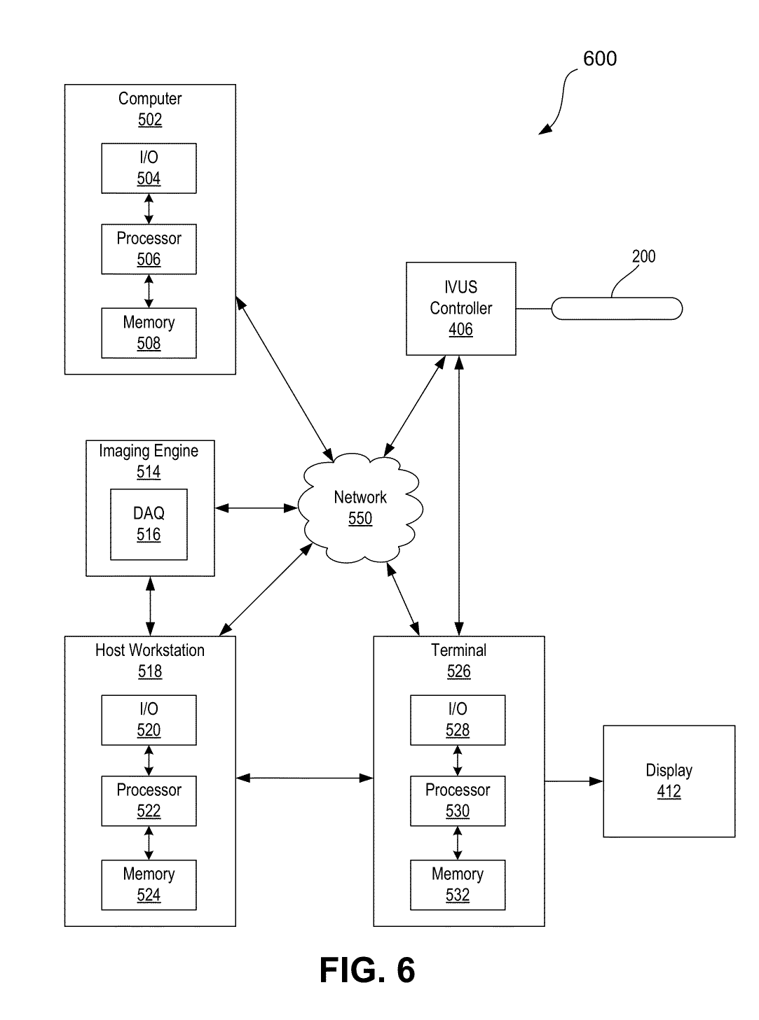

A system of the invention may be implemented in a number of formats. An embodiment of a system 500 of the invention is shown in FIG. 5. The core of the system 500 is a computer 502 or other computational arrangement comprising a processor 506 and memory 508. The memory has instructions which when executed cause the processor to determine a baseline measurement prior to conducting a therapeutic procedure and determine a post-therapy measurement after conducting the therapeutic procedure. The instructions may also cause the computer to compare the post-therapy measurement to the baseline measurement, thereby determining the degree of post-therapy improvement after conducting the therapeutic procedure. In the system of the invention, the physiological measurement data of vasculature will originate with an IVUS imaging probe 200 as discussed above, whose acquired data sets may be collected with IVUS controller 406. Having collected the image data, the processor then processes the data to build images and identify flow and/or structures and then outputs the results. The results are typically output to a display 412 to be viewed by a physician or technician.

In advanced embodiments, system 500 may comprise an imaging engine 510 which has advanced image processing features, such as image tagging, that allow the system 500 to more efficiently process and display intravascular and angiographic images. The imaging engine 510 may automatically highlight or otherwise denote areas of interest in the vasculature. The imaging engine 510 may also produce 3D renderings or other visual representations of the physiological measurements. In some embodiments, the imaging engine 510 may additionally include data acquisition functionalities (DAQ) 512, which allow the imaging engine 510 to receive the physiological measurement data directly from the guidewire 100 to be processed into images for display.

Other advanced embodiments use the I/O functionalities 504 of computer 502 to control the IVUS controller for the IVUS probe 200. While not shown here, it is also possible that computer 502 may control a manipulator, e.g., a robotic manipulator, connected to IVUS probe 200 to improve the placement of the probe 200.

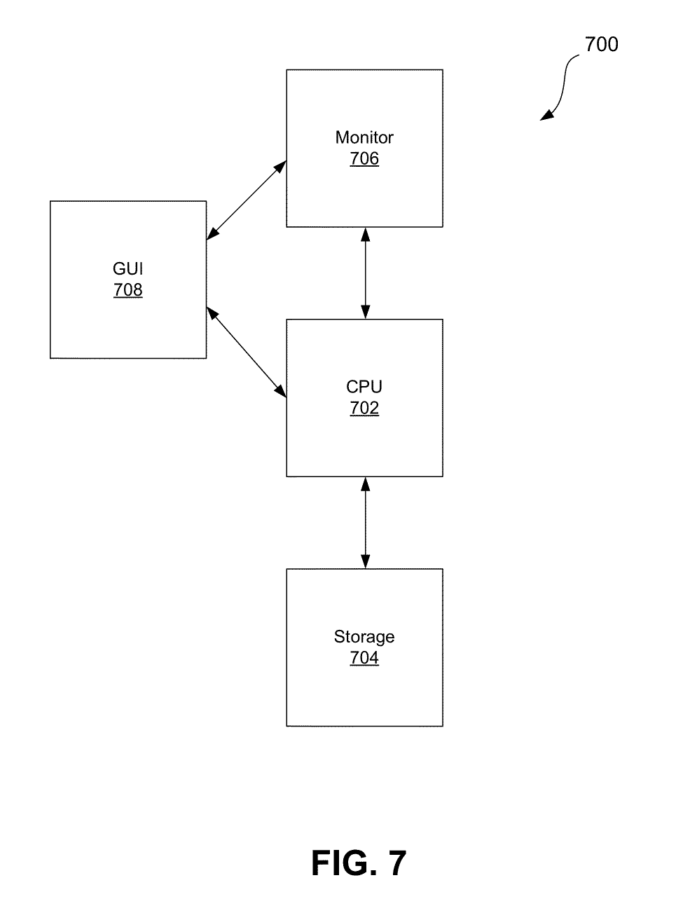

A system 600 of the invention may also be implemented across a number of independent platforms which communicate via a network 550, as shown in FIG. 6. FIG. 6 is a rendition of the block diagram of FIG. 5 depicting how data can be stored and transferred over a hospital (internal and external) network and then accessed by a user via a separate terminal also connected to the same network. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 6, the IVUS controller 406 may facilitate obtaining the data, however the actual implementation of the steps can be performed by multiple processors working in communication via the network 550, for example a local area network, a wireless network, or the internet. The components of system 600 may also be physically separated. For example, terminal 526 and display 412 may not be geographically located with the IVUS controller 406.

As shown in FIG. 6, imaging engine 514 communicates with host workstation 518 over network 550. In some embodiments, an operator uses host workstation 518 or terminal 526 to control system 600 or to receive images. An image may be displayed using an I/O 504, 520, 528, which may include a monitor. Any I/O may include a monitor, keyboard, mouse, or touch screen to communicate with any of processor 506, 522, or 530, for example, to cause data to be stored in any tangible, non-transitory memory 508, 524, or 532. Input from a user is received by a processor in an electronic device such as, for example, host workstation 518. In certain embodiments, host workstation 518 and imaging engine 514 are included in a bedside console unit to operate system 600.

In some embodiments, the system may render three dimensional imaging of the vasculature or the intravascular images. An electronic apparatus within the system (e.g., PC, dedicated hardware, or firmware) such as the host workstation 518 stores the three dimensional image in a tangible, non-transitory memory and renders an image of the 3D tissues on the display 412. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 518. The selection can be rendered into a visible display. In some embodiments, an operator uses host workstation 518 to control system 600 or to receive images.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 518 and imaging engine 516 are included in a bedside console unit to operate system 600.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network 550 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 550 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 412 is rendered within a computer operating system environment, such as Windows, Mac OS, or Linux or within a display or GUI of a specialized system. Display 412 can include any standard controls associated with a display (e.g., within a windowing environment) including minimize and close buttons, scroll bars, menus, and window resizing controls. Elements of display 412 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 412 can further include any controls or information related to viewing images (e.g., zoom, color controls, brightness/contrast) or handling files comprising three-dimensional image data (e.g., open, save, close, select, cut, delete, etc.). Further, display 412 can include controls (e.g., buttons, sliders, tabs, switches) related to operating a three dimensional image capture system (e.g., go, stop, pause, power up, power down).

In certain embodiments, display 412 includes controls related to three dimensional imaging systems that are operable with different imaging modalities. For example, display 412 may include start, stop, zoom, save, etc., buttons, and be rendered by a computer program that interoperates with IVUS, OCT, or angiogram modalities. Thus display 412 can display an image derived from a three-dimensional data set with or without regard to the imaging mode of the system.

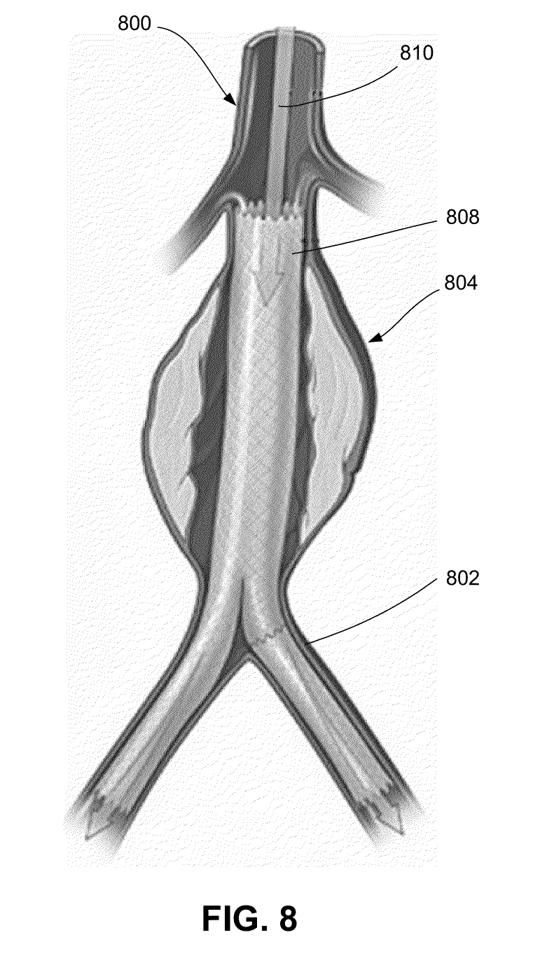

Alternatively, an imaging data set may be assessed, analyzed, and transformed with a system such as the system shown in FIG. 7, comprising CPU 702, storage 704, and monitor 706. Storage 704 may contain instructions for carrying out methods of the invention, e.g., to configure CPU 702 to analyze the imaging data set for a parameter, assign an indicator to the medical device based on the presence of the parameter, and display the indicator on monitor 706. For example CPU 702 may direct monitor 706 to display a longitudinal image of a lumen with a color-coded stent. In some embodiments, a system of the invention will additionally comprise graphical user interface (GUI) 708, which allows a user to interact with the images. In some embodiments, CPU 702, storage 704, and monitor 706 may be encompassed within system 700.

The systems and methods of use described herein can be performed using any type of computing device, such as a computer, that includes a processor or any combination of computing devices where each device performs at least part of the process or method. In some embodiments, systems and methods described herein may be performed with a handheld device, e.g., a smart tablet, or a smart phone, or a specialty device produced for the system.

In some embodiments, a device of the invention includes an OCT imaging system and obtains a three-dimensional data set through the operation of OCT imaging hardware. In some embodiments, a device of the invention is a computer device such as a laptop, desktop, or tablet computer, and obtains a three-dimensional data set by retrieving it from a tangible storage medium, such as a disk drive on a server using a network or as an email attachment.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Any target can be imaged by methods and systems of the invention including, for example, bodily tissue. In certain embodiments, systems and methods of the invention image structural information and movement or flow within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

The IVUS probe 200 and systems 100, 400, 500, 600 and 700 of the present invention may be used to detect, and further identify, endoleaks associated with aneurysm repair procedures, such as EVAR or TEVAR procedures. Reference will now be made to endovascular aneurysm repair (EVAR) procedure. Methods of the invention are useful with all EVAR related procedures, including without limitation, EVAR, hybrid EVAR, Common Iliac Artery EVAR, and Thoracic EVAR (TEVAR).

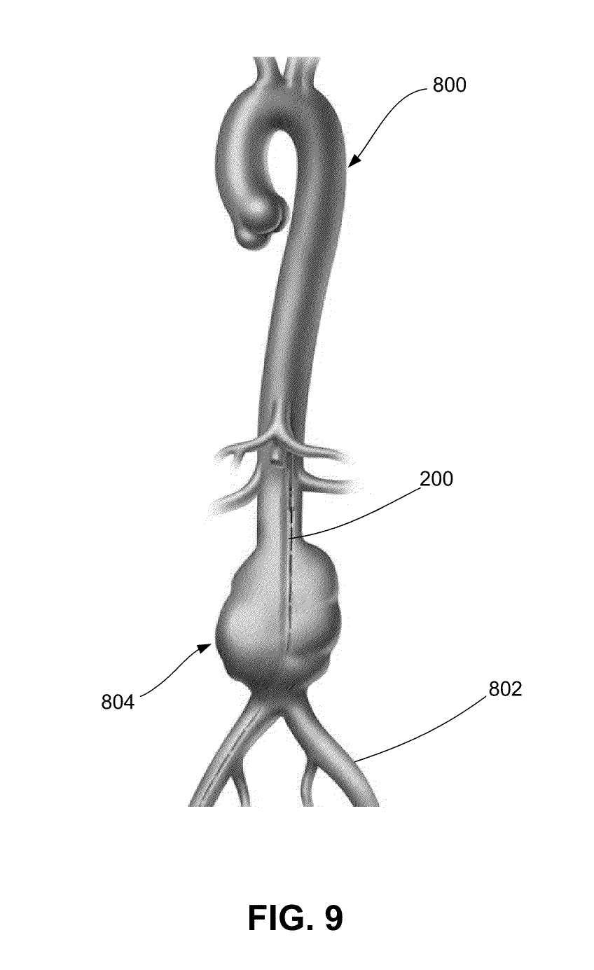

FIG. 8 is a cross-sectional view of a portion of an aorta 800 illustrating placement of stent graft 808 at an abdominal aortic aneurysm (AAA) site 804 during an EVAR procedure. EVAR is typically conducted in a sterile environment, usually a theatre, under x-ray fluoroscopic guidance. The patient is usually administered an anesthetic prior to conducting the procedure. A puncture is then made with a needle in the femoral artery 802 of the groin. An introducer or vascular sheath is then inserted into the artery with a large needle, and after the needle is removed, the introducer provides access for guidewires, catheters, and other endovascular tools, such as the stent graft 308 used to treat the abdominal aneurysm 804. As shown, once in place, the stent graft 808 acts as an artificial lumen for blood to flow through, as indicated by arrow 810, and not into the surrounding aneurysm sac 804. This reduces the pressure in the aneurysm 804, which itself will usually thrombose and shrink in size over time.

Diagnostic angiography images or ‘runs’ of the aorta are captured to determine the location of the patient's renal arteries, so the stent graft can be deployed without blocking them. Blockage may result in renal failure, thus the precision and control of the graft stent deployment is extremely important. The main ‘body’ of the stent graft is placed first, follow by the ‘limbs’ which join on to the main body and sit on the Aortic Bifurcation for better support, and extend to the Iliac arteries. The stent graft (covered stent), once positioned, serves as an artificial lumen for blood to flow down, and not into the surrounding aneurysm sac. Accordingly, pressure is taken off the aneurysm wall, which itself will thrombose in time.

For certain occasions that the aneurysm extends down to the Common Iliac Arteries, a specially designed graft stent, named as Iliac Branch Device (IBD), can be used, instead of blocking the Internal Iliac Arteries, but to preserve them. The preservation of the Internal Iliac Arteries is important to prevent Buttock Claudication, and to preserve the full genital function.

A variation of EVAR is the Hybrid Procedure. A hybrid procedure occurs in the angiography room and aims to combine endovascular procedures with limited open surgery. In this procedure the stent graft deployment is planned to combine with an open operation to revascularise selected arteries that will be “covered” by the stent graft i.e. deprived of arterial inflow. In this method more extensive EVAR devices can be deployed to treat the primary lesion while preserving arterial flow to critical arteries.

Thoraco-abdominal aneurysms (TAA) typically involve such vessels and deployment of the EVAR device will cover important arteries e.g. visceral or renal arteries, resulting in end organ ischemia which may not be survivable. The open operation component aims to bring a bypass graft from an artery outside the stent graft coverage to vital arteries within the coverage region. This component adds to the EVAR procedure in time and risk but is usually judged to be lesser that the risk of the major totally open operation.

The above procedures aim to reduce the morbidity and mortality of treating certain types of arterial disease. The occurrence of endoleaks, however, can significantly increase the risk associated with EVAR procedures. An endoleak is characterized by persistent blood flow within the aneurysm sac following endovascular aneurysm repair. Normally the aortic stent graft used for EVAR excludes the aneurysm from the circulation by providing a conduit for blood to bypass the sac. An improperly positioned or defective graft, however, can result in an ineffectual seal and result in the formation of endoleaks.

An endoleak is a common complication of EVAR and is found in a significant number of patients intraoperatively (seen on the on-table angiogram after stent deployment), as well as during follow-up. This somewhat common occurrence greatly reduces the overall effectiveness of the EVAR procedure. Although some endoleaks appear to be unavoidable due to the presence of pre-existing patent branch vessels arising from the aneurysm sac, others occur as a result of poor graft selection.

In either situation, there is an immediate need to monitor the occurrence of endoleaks, preferably during the procedure itself (perioperatively). Systems and methods of the invention address this need and can be used perioperatively. While the patient is still on the operating table and has the introducer used for delivering the stent graft still inside him, the same introducer can be used to maneuver the IVUS probe 200 to the site of the implanted graft and acquire image data near the site of implantation to further provide flow visualization for the detection and characterization of endoleaks.

Endoleaks are typically classified as type I, type II, type III, type IV, and type V endoleaks.

Type I endoleaks occur as a result of an inadequate seal at the site of the graft attachment. It may occur at the proximal end or distal end. Blood flow leaks alongside the graft into the aneurysm sac. They are often the result of unsuitable patient (aneurysm) selection or device selection for the EVAR procedure, but can also occur if the graft migrates. Type I leaks are always considered significant as they do not tend to resolve spontaneously.

Type II endoleaks are the most common. In this situation, retrograde flow though branch vessels continues to fill the aneurysm sac. The most common culprit vessels are lumbar arteries, inferior mesenteric artery or internal iliac artery. This type of leak has been a substantial number of cases. It usually resolves spontaneously over time and requires no treatment. Embolization of the branch vessel is indicated if the aneurysm sac continues to expand in size.

Type III endoleaks are caused by mechanical failure of the stent graft. There may be a fracture of the stent graft, hole or defect on the graft fabric, or junctional separation of the modular components. Causes may relate to defective device material, extreme angulation of a segment predisposing to fracture, or improper overlap of the modular components during insertion.

Type IV endoleaks occur when blood leaks across the graft due to its porosity. It does not require any treatment and typically resolves within a few days of graft placement.

Type V “leak” (also referred to as endotension) is not a true leak but is defined as continued expansion of the aneurysm sac without evidence of a leak site. It is also referred to as endotension. Its origin is still unclear but is believed to be due to pulsation of the graft wall with transmission of the pulse wave through the perigraft space (aneurysm sac) to the native aneurysm wall.

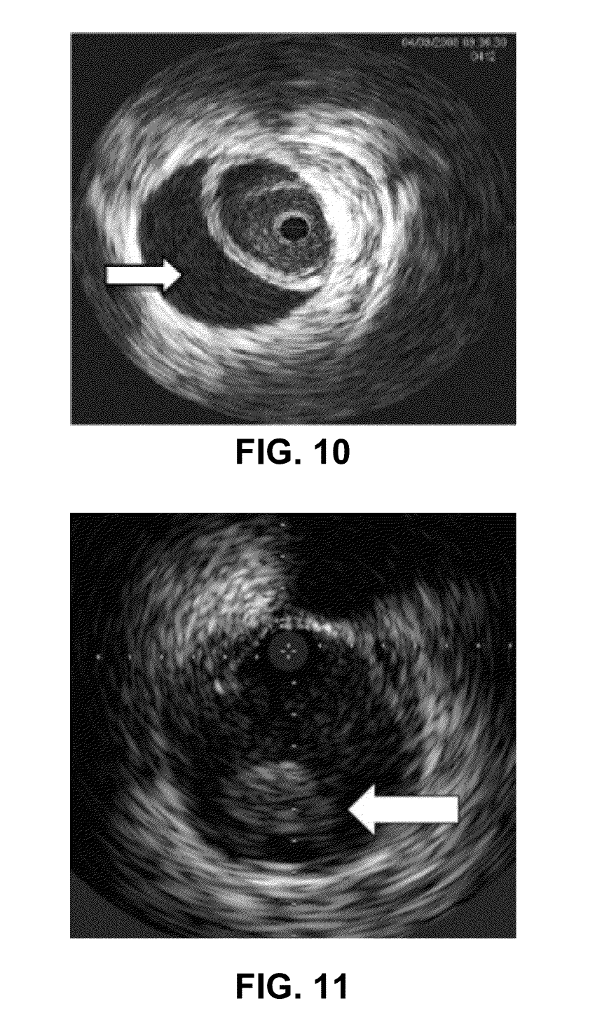

FIG. 9 is a side view of the aorta 800 of FIG. 8 illustrating positioning of an IVUS probe 200 for capturing image data related to at least the stent graft 808 and aortic wall for providing at least flow visualization for the detection and further classification of endoleaks. In one embodiment, systems of the present invention are configured to provide automated detection and classification of at least types I, II, III, and IV endoleaks based on the flow visualization provided by captured and processed image data, in accordance with methods described herein.

During a procedure, such as EVAR or TEVAR, a surgeon may perform a manual or automated pullback of the IVUS transducer elements 218, for example, of the probe 200, so as to capture image data and further visualize the aortic anatomy, as well as the stent graft at the site of an abdominal aortic aneurysm (AAA). Systems described and illustrated in FIGS. 1 and 4-7 are further configured to provide detection of at least one of a false lumen, mal-apposition, as well as graft enfolding and other features identifiable with gray-scale and color flow, and further provide automatic identification of a site-of-interest with a potential endoleak.

Furthermore, the systems described and illustrated in FIGS. 1 and 4-7 are further configured to automatically classify a detected endoleak as one of types I, II, III, or IV based on the flow visualization data, as well as data related to the surrounding tissue (e.g., aortic wall, stent graft, etc.). For example, in one embodiment, systems may be configured to detect mal-apposition and/or graft enfolding based on attributes of the lumen (e.g., dimensions of the aorta) before and after placement of the stent graft so as to determine if the endoleak is the distal or proximal landing zone of the graft. Next, the system is configured to confirm leakage by cross-referencing color flow data, which may include motion detection. In turn, the system is configured to determine that the endoleak is a type IA (proximal) or type IB (distal) endoleak.

In the event that no mal-apposition and/or graft enfolding is detected, the system is configured to search image data for dual flow channels having different directions in the presence of the stent graft. In the event that dual flow channels having different directions are detected, the system is configured to determine that the endoleak is a type III endoleak. However, if the presence of a dual flow channel having different directions is detected in absence of a stent graft, the GUI 708, for example, is configured to provide a warning to the surgeon that there is possibly a dissection in the aortic wall, at which point the surgeon can act accordingly to prevent further bleeding and possible death.

In the event that no dual or multiple flow channels are detected from within the luminal surface of the aorta (or vessel of interest), the system is further configured to search image data for other flow channels and associated direction of flow. In the event that flow channels are detected at or near the outer rim of the AAA, the system is configured to determine that the endoleak is a type II endoleak.

In the event that no flow channels or mal-appositions are detected, the system is configured to search data for flow within the lumen or within the AAA (in absence of a stent graft) for areas with flow in the same direction without dual lumens, but with different flow rates and/or with “plaque” appearing within the lumen, which may be indicative of a thrombus.

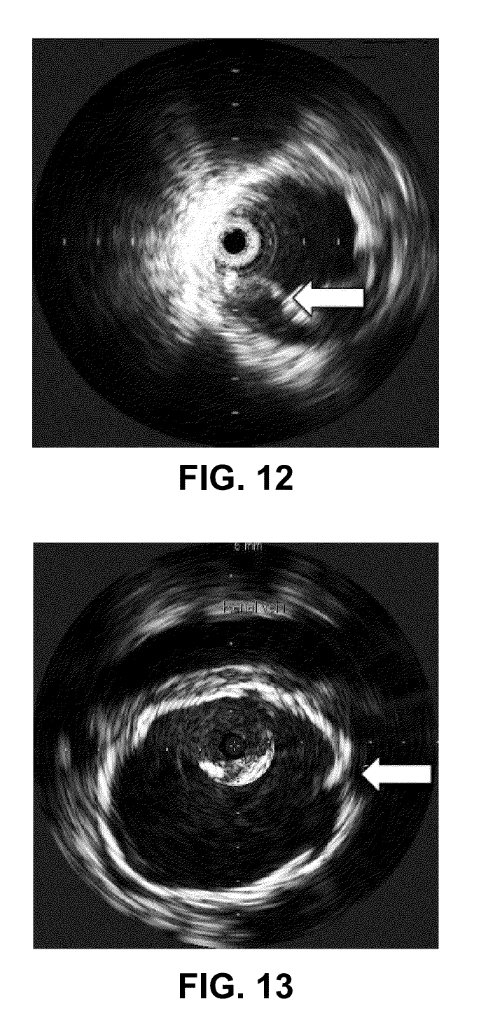

A system of the invention is capable of imaging a biological lumen, assessing properties of the lumen, and then displaying the collected information in an easy-to-read format. For example, FIGS. 10-13 show various gray-scale IVUS images of vessels provided by a system consistent with the present disclosure. The gray-scale images can be produced based on image data acquired during the gray-scale imaging mode, for example, of the IVUS transducers. However, as seen in FIGS. 10-13, structural data is conveyed.

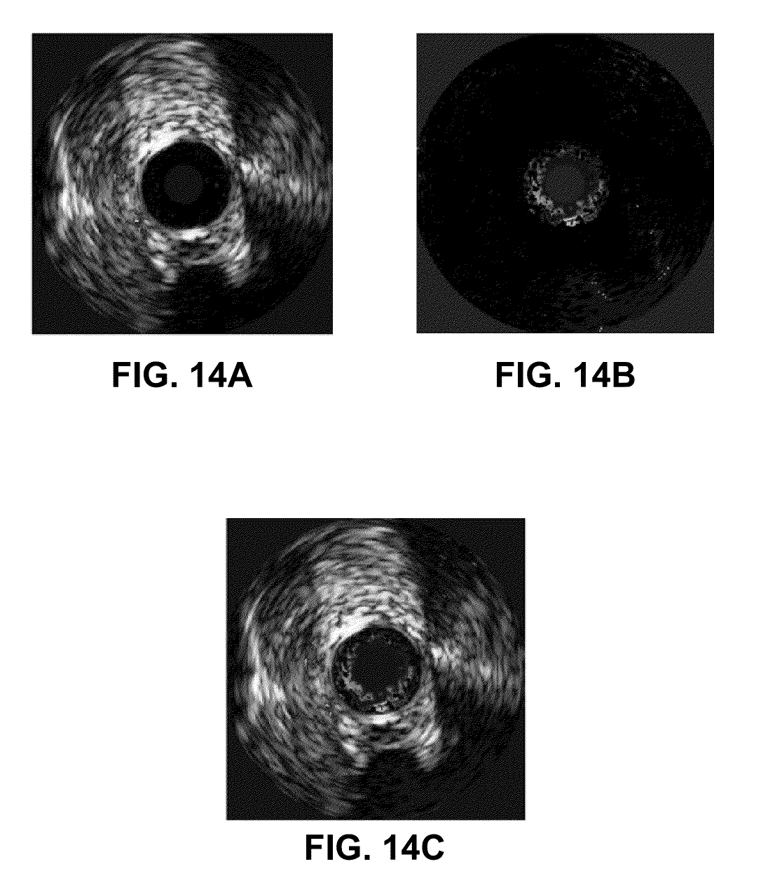

As described herein, a system of the invention is capable of evaluating the structural information within a blood vessel based at least on flow data. FIG. 14A is a gray-scale IVUS image of a vessel, FIG. 14B is an image of flow within the vessel, and FIG. 14C is a composite image of the flow data overlaid on the gray-scale image provided by a system consistent with the present invention. As shown in FIGS. 7A-7C, the flow data can be overlaid with the image data to provide a combined image. FIGS. 7A-7C depict 360 degree cross-section views of the inside of a vessel and the flow data represents blood flow in the vessel. The combined image provides an additional level of detail to a physician that is not provided by any IVUS image alone. FIG. 7A shows a gray-scale image alone, while FIG. 7B shows an image of flow data alone. FIG. 7C shows an overlay of the image of the flow data on the gray-scale image. It should be appreciated that blood-vessel data can be used in a number of applications including, but not limited to, diagnosing and/or treating patients. For example, blood-vessel data can be used to identify and/or image blood vessel borders or boundaries, as provided by U.S. Pat. No. 6,381,350, which is incorporated by reference herein in its entirety. Another use for blood-vessel data is for classifying and/or imaging vascular plaque, as provided by U.S. Pat. No. 6,200,268, which is also incorporated by reference herein in its entirety. Another use for blood-vessel data is to classify vascular tissue, as provided by U.S. Pat. No. 8,449,465, which is also incorporated by reference herein in its entirety.

In some embodiments, the images will be displayed in real time and may oscillate in color or shade to communicate information regarding flow, velocity, or direction, among other information. In alternative embodiments, the system can be used to evaluate the placement of a device, such as a stent. Using a system of the invention, an IVUS image of a portion of a vessel with a placed stent is collected with an intravascular imaging probe, such as IVUS probe 200, and the image processing components produce an image showing a cut-away of the vessel including arms of a stent. For example, as shown in FIG. 13, a stent is mal-apposed or enfolded, i.e., portions of the stent are not touching the luminal wall. Mal-apposed stents can further exacerbate cardiovascular issues because the pocket between the lumen wall and the stent fills with plaque or thrombus, greatly reducing blood flow through the region.

Methods of the invention can further encompass treatment of the endoleak upon detection based on functional parameters. Treatment will depend on the type of endoleak.

Type I leaks are generally treated as soon as detected. Extension cuffs or covered stents can be inserted at the leaking graft end to improve the seal, or embolization of the leak site with glue or coils can be used. Rarely, if detected intra-operatively during EVAR, conversion to an open procedure may be required if endovascular methods of sealing the leak are unsuccessful.

Type II leaks (retrograde flow through branch) usually spontaneously thrombose. As such at many institutions these leaks are not treated immediately; watchful waiting is employed and if the leak persists it is treated by embolising the branch vessel with glue or coils. Pre-emptive embolization of potential sources of collateral flow is sometimes performed prior to stent-graft insertion, particularly the internal iliac artery in select cases. Pre-emptive embolization of other branch vessels is controversial.

Type III leaks (graft mechanical failure) do not spontaneously resolve and are therefore treated immediately, usually with additional stent-graft components.

Type IV leaks (graft porosity) cannot be treated except by improving device selection.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations. Steps of the invention may be performed using dedicated medical imaging hardware, general purpose computers, or both. As one skilled in the art would recognize as necessary or best-suited for performance of the methods of the invention, computer systems or machines of the invention include one or more processors (e.g., a central processing unit (CPU) a graphics processing unit (GPU) or both), a main memory and a static memory, which communicate with each other via a bus. A computer device generally includes memory coupled to a processor and operable via an input/output device.

Exemplary input/output devices include a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory according to the invention can include a machine-readable medium on which is stored one or more sets of instructions (e.g., software), data, or both embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any of the methodologies of the present invention. The term “machine-readable medium” shall accordingly be taken to include, but not be limited to, solid-state memories (e.g., subscriber identity module (SIM) card, secure digital card (SD card), micro SD card, or solid-state drive (SSD)), optical and magnetic media, and any other tangible storage media. Preferably, computer memory is a tangible, non-transitory medium, such as any of the foregoing, and may be operably coupled to a processor by a bus. Methods of the invention include writing data to memory—i.e., physically transforming arrangements of particles in computer memory so that the transformed tangible medium represents the tangible physical objects—e.g., the arterial plaque in a patient's vessel.

As used herein, the word “or” means “and or or”, sometimes seen or referred to as “and/or”, unless indicated otherwise.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for an intraluminal procedure, said system comprising:
a sensor operable to capture image data of a vessel; and
at least one processor configured to:
receive and process said image data;
provide output representing a cross-section of said vessel and a flow characteristic of a fluid therein based on said processed image data; and
detect the presence of an endoleak within said vessel based on said flow characteristic.

2. The system of claim 1, wherein said processor is configured to:
identify one or more attributes of said endoleak; and
classify said endoleak as at least one of type I, type II, type III, or type IV leak based, at least in part, on said one or more attributes.

3. The system of claim 2, wherein said one or more attributes include direction of flow, velocity of flow, and location of flow in relation to a wall of said vessel.

4. The system of claim 1, wherein said endoleak is associated with endovascular aneurysm repair.

5. The system of claim 4, wherein said processor is configured to detect an interventional structure within said vessel associated with said aneurysm repair.

6. The system of claim 5, wherein said interventional structure comprises a stent graft.

7. The system of claim 6, wherein said processor is configured to identify one or more attributes of said stent graft.

8. The system of claim 7, wherein said one or more attributes are selected from the group consisting of dimensions, location within the vessel in relation to the aneurysm, and location relative to the vessel wall.

9. The system of claim 8, wherein said processor is configured to classify said endoleak as at least one of type I, type II, type III, or type IV based, at least in part, on said one or more attributes of said endoleak and attributes of said stent graft.

10. The system of claim 1, wherein said sensor comprises an intravascular ultrasound (IVUS) probe.

11. The system of claim 10, wherein said IVUS probe is operable to capture said intraluminal data via ultrasound at a frequency less than 15 MHz.

12. The system of claim 11, wherein said frequency is between 9 MHz and 11 MHz.

13. The system of claim 1, wherein said vessel comprises an aortic vessel.

14. The system of claim 13, wherein said vessel is an abdominal aortic vessel.

15. The system of claim 13, wherein said vessel is a thoracic aortic vessel.

16. The system of claim 2, wherein said endovascular aneurysm repair-based procedure is selected from standard endovascular aneurysm repair (standard EVAR), thoracic endovascular aneurysm repair (TEVAR), Hybrid EVAR, or Iliac Artery EVAR.

17. A method for detecting and classifying endoleaks associated with endovascular aneurysm repair, the method comprising:
inserting a sensor into a vessel;
capturing image data of said vessel;
processing said image data;
providing output representing a cross-section of said vessel and a flow characteristic of a fluid therein based on said processed image data; and
detecting the presence of an endoleak within said vessel based on said flow characteristic.

18. The method of claim 17, further comprising:
identifying one or more attributes of said endoleak; and
classifying said endoleak as at least one of type I, type II, type III, or type IV leak based, at least in part, on said one or more attributes.

19. The method of claim 18, wherein said one or more attributes include direction of flow, velocity of flow, and location of flow in relation to a wall of said vessel.

20. The method of claim 18, further comprising:
detecting an interventional structure within said vessel associated with said aneurysm repair; and
identifying one or more attributes of said stent graft; and
classifying said endoleak as at least one of type I, type II, type III, or type IV based on said attributes of said endoleak and said interventional structure.

21. The method of claim 20, wherein said interventional structure comprises a stent graft.

22. The method of claim 20, wherein said one or more attributes are selected from the group consisting of dimensions, location within the vessel in relation to the aneurysm, and location relative to the vessel wall.
